(19) United States
(12) Patent Application Publication
(10) Pub. No.: US 2026/0283624 A1
(43) Pub. Date: Sep. 24, 2026

(54) SURGICAL CUTTING GUIDE

(71) Applicant: JHNB LLC, Wyomissing, PA (US)

(72) Inventor: Eric Slotkin, Wyomissing, PA (US)

(21) Appl. No.: 19/083,882

(22) Filed: Mar. 19, 2025

(51) Int. Cl. A61B 17/15; A61B 17/00; A61B 34/10

(52) U.S. Cl. CPC A61B 17/155; A61B 34/10; A61B 2017/00526; A61B 2034/105

(57) ABSTRACT

A customized surgical cutting guide including a non-metallic base and a first cutting guide surface, a second cutting guide surface, a third cutting guide surface, a fourth cutting guide surface and a fifth cutting guide surface formed on the non-metallic base. The cutting guide further includes a medial opening and a lateral opening about a distal end thereof, wherein each of the medial and lateral openings is sized sufficiently to receive a peg post of a femoral implant component.

### FIELD OF THE INVENTION

The subject disclosure relates generally to surgical devices and, more particularly, to a cutting guide for preparing a bone to receive an implant.

### BACKGROUND OF THE INVENTION

When performing a total knee arthroplasty (TKA) procedure, both the femur and tibia must be cut to receive the applicable knee implant components e.g., a femoral implant and a tibial implant, respectively. For the femur, typically five major bone cuts are made to the distal femur to enable it to receive the femoral implant. This can be done with a number of different types of bone cutting guides that allow for individual cuts of one or more of the five major bone cuts, or a single cutting guide that enables all five major bone cuts to be made. Such cutting guides are also typically sized to match up with the planned size of the knee implant itself. However, additional cutting guides are necessary to make additional bone cuts to the distal femur in order to allow for all the various components of a given femoral implant to be seated properly on the distal femur. Such bone cuts are made, for example via a femoral peg hole cutting guide to make e.g., a counterbore or blind hole, within the distal femur to receive respective femoral pegs on a femoral implant. Additionally, a punch guide instrument may be needed to cut out a notch within the distal femur if the femoral implant to be implanted requires a box cut for a femoral implant box of a posterior stabilized femoral implant or clearance for post of a tibial insert of a posterior stabilized bearing insert.

Thus, while conventional instrumentation exists to allow for the preparation of the various necessary bone cuts on a distal femur to accept a femoral implant, it requires the attachment and removal of multiple cutting guides which can lead to misalignment issues with the various cutting guides and therefore misaligned bone cuts and increases in overall operation time (and consequently increases in patient time under anesthesia). Accordingly, a need still exists for a more convenient cutting guide system that addresses the foregoing issues.

### BRIEF SUMMARY OF THE INVENTION

The subject disclosure provides a customized surgical cutting guide comprising: a non-metallic base having a patient-specific bone engaging surface; a first cutting guide surface, a second cutting guide surface, a third cutting guide surface, a fourth cutting guide surface and a fifth cutting guide surface formed on the non-metallic base; a medial opening about a distal end of the non-metallic base sized sufficiently to receive a peg post of a femoral implant component; and a lateral opening about the distal end of the non-metallic base sized sufficiently to receive a peg post of a femoral implant component. The first cutting guide surface, the second cutting guide surface, the third cutting guide surface, the fourth cutting guide surface, and the fifth cutting guide surface are positioned on the non-metallic base based on patient-specific 3D bone models of a patient.

The non-metallic base includes a detachable center portion. The detachable center portion is sized sufficiently to receive a femoral preparation instrument therethrough. The customized surgical cutting guide can further comprise a femoral punch guide for replacing the detachable center portion.

The non-metallic base is a unitarily formed base. The medial and lateral openings are distally facing openings.

The non-metallic base comprises a polymer. The non-metallic base comprises polyethylene terephthalate (PET), polyethylene terephthalate glycol (PETG), polycarbonate (PC), polypropylene (PP), nylon, polyetheretherketone (PEEK), polyetherketoneketone (PEKK), polyetherimide (PEI), acrylonitrile styrene acrylate (ASA), and/or acrylonitrile butadiene styrene (ABS) and polylactic acid (PLA).

The subject disclosure further discloses a method of manufacturing a patient-specific, non-metallic surgical cutting guide comprising: obtaining a medical image of a first bone and a second bone of a joint of a patient; rendering 3D models of the first and second bones of the joint based on the medical image; determining relative positions of the 3D models of the first and second bones relative to each other for establishing joint balancing; determining positions for a first bone cut, a second bone cut, a third bone cut, a fourth bone cut, and a fifth bone cut to the first bone based on the determined relative positions of the 3D models of the first and second bones; and manufacturing a non-metallic surgical cutting guide having: a bone engaging surface based on the rendered 3D model of the first bone, a first cutting guide surface, a second cutting guide surface, a third cutting guide surface, a fourth cutting guide surface and a fifth cutting guide surface each positioned based on the determined positions for the first bone cut, the second bone cut, the third bone cut, the fourth bone cut, and the fifth bone cut, a medial opening about its distal end sized sufficiently to receive a peg post of a femoral implant component, and a lateral opening about its distal end sized sufficiently to receive a peg post of a femoral implant component. The non-metallic surgical cutting guide further includes a detachable center portion. The method can further include 3D printing the surgical cutting guide.

The subject disclosure further discloses a surgical cutting guide comprising: a base having a bone engaging surface; a first cutting guide surface, a second cutting guide surface, a third cutting guide surface, a fourth cutting guide surface and a fifth cutting guide surface formed on the base; a medial opening about a distal end of the non-metallic base sized sufficiently to receive a peg post of a femoral implant component; and a lateral opening about the distal end of the non-metallic base sized sufficiently to receive a peg post of a femoral implant component.

The base includes a detachable center portion. The detachable center portion is sized sufficiently to receive a femoral preparation instrument therethrough. The surgical cutting guide further comprises a femoral punch guide for replacing the detachable center portion. The medial and lateral openings are distally facing openings.

### BRIEF DESCRIPTION OF THE SEVERAL VIEWS OF THE DRAWINGS

The foregoing summary, as well as the following detailed description of the exemplary embodiments of the subject disclosure, will be better understood when read in conjunction with the appended drawings. For the purpose of illustrating the subject disclosure, there are shown in the drawings exemplary embodiments. It should be understood, however, that the subject disclosure is not limited to the precise arrangements and instrumentalities shown.

In the drawings:

FIG. 1 is a perspective view of a conventional femoral implant component;

FIG. 2 is front view of a patient-specific cutting guide in accordance with a first exemplary embodiment of the subject disclosure;

FIG. 3 is a perspective view of the patient-specific cutting guide of FIG. 2;

FIG. 4 is a front view of a patient-specific cutting guide in accordance with a further exemplary embodiment of the subject disclosure;

FIG. 5 is perspective view of a femoral punch guide applicable to the embodiments of the subject disclosure; and

FIG. 6 is a front view of a patient-specific cutting guide in accordance with a another exemplary embodiment of the subject disclosure.

### DETAILED DESCRIPTION

Reference will now be made in detail to the various embodiments of the subject disclosure illustrated in the accompanying drawings. Wherever possible, the same or like reference numbers will be used throughout the drawings to refer to the same or like features. It should be noted that the drawings are in simplified form and are not drawn to precise scale. Certain terminology is used in the following description for convenience only and is not limiting. Directional terms such as top, bottom, left, right, above, below and diagonal, are used with respect to the accompanying drawings. The term “distal” shall mean away from the center of a body. The term “proximal” shall mean closer towards the center of a body and/or away from the “distal” end. The words “inwardly” and “outwardly” refer to directions toward and away from, respectively, the geometric center of the identified element and designated parts thereof. Such directional terms used in conjunction with the following description of the drawings should not be construed to limit the scope of the subject disclosure in any manner not explicitly set forth. Additionally, the term “a,” as used in the specification, means “at least one.” The terminology includes the words above specifically mentioned, derivatives thereof, and words of similar import.

“About” as used herein when referring to a measurable value such as an amount, a temporal duration, and the like, is meant to encompass variations of ±20%, ±10%, ±5%, ±1%, or ±0.1% from the specified value, as such variations are appropriate. “Substantially” as used herein shall mean considerable in extent, largely but not wholly that which is specified, or an appropriate variation therefrom as is acceptable within the field of art. “Exemplary” as used herein shall mean serving as an example.

Throughout this disclosure, various aspects of the subject disclosure can be presented in a range format. It should be understood that the description in range format is merely for convenience and brevity and should not be construed as an inflexible limitation on the scope of the subject disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Furthermore, the described features, advantages and characteristics of the exemplary embodiments of the subject disclosure may be combined in any suitable manner in one or more embodiments. One skilled in the relevant art will recognize, in light of the description herein, that the present disclosure can be practiced without one or more of the specific features or advantages of a particular exemplary embodiment. In other instances, additional features and advantages may be recognized in certain embodiments that may not be present in all exemplary embodiments of the subject disclosure.

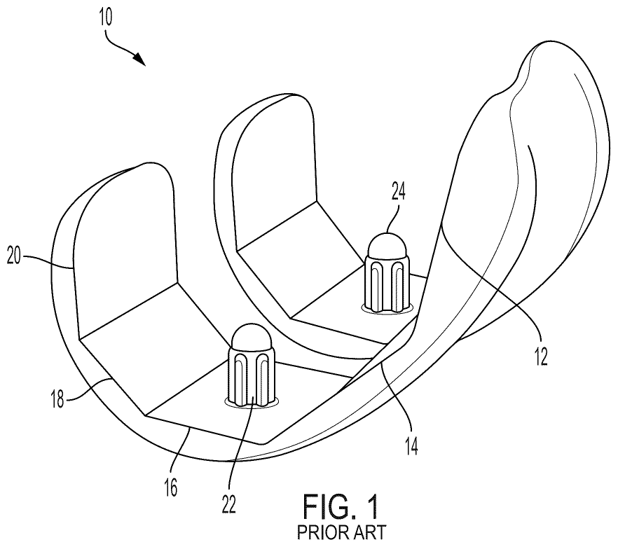

Referring to FIG. 1 there is shown a perspective view of a conventional cruciate retaining (CR) femoral implant 10. The interior surfaces or bone engaging surfaces of the femoral implant 10 includes five substantially planar surfaces, an anterior surface 12, an anterior chamfer surface 14, a distal surface 16, a posterior chamfer surface 18, and a posterior surface 20. The femoral implant also includes a medial peg post 22 and a lateral peg post 24 that extend upwardly from the distal surface 16.

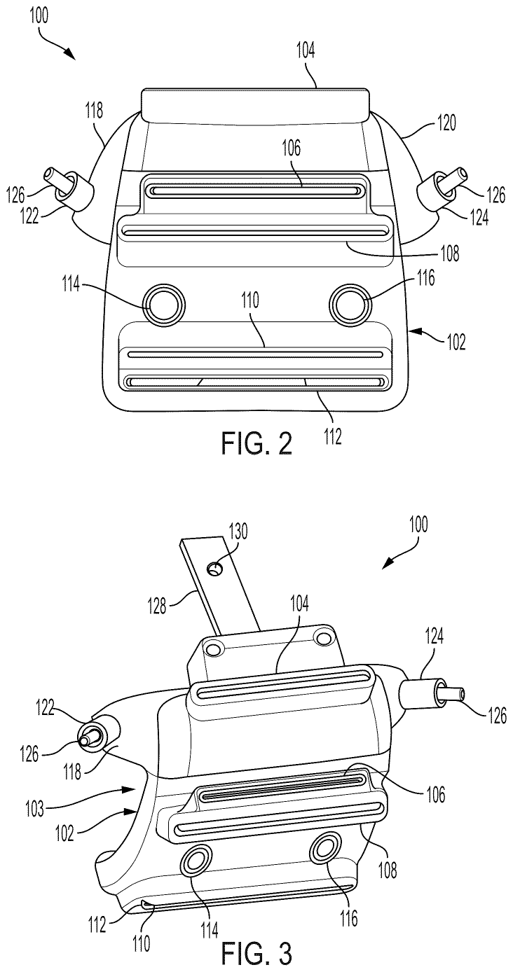

Referring to FIGS. 2 and 3, there is shown a patient-specific cutting guide or customized surgical cutting guide 100 in accordance with a first exemplary embodiment of the subject disclosure. The cutting guide 100 includes a base e.g., a non-metallic base 102 which can be a unitarily formed base. The non-metallic base may be formed by a number of forming processes, such as, a molding process, the use of mold forms, or a three-dimensional (3D) printing process.

The non-metallic base is formed out of a non-metal, such as a polymer or a plastic. The non-metallic base is formed out of a non-ceramic. That is the non-metallic base is a polymer-based base or a plastic base. The non-metallic base can be formed of or comprised of, without limitation, polyethylene terephthalate (PET), polyethylene terephthalate glycol (PETG), polycarbonate (PC), polypropylene (PP), nylon, polyetheretherketone (PEEK), polyetherketoneketone (PEKK), polyetherimide (PEI), acrylonitrile styrene acrylate (ASA), acrylonitrile butadiene styrene (ABS), polylactic acid (PLA) and/or the like.

The non-metallic base 102 includes a patient-specific bone engaging surface 103. The patient-specific bone engaging surface is the surface of the cutting guide that comes in contact with and/or correspondingly mates with the bone geometry of the patient's distal femur, thereby allowing for a matched or matingly engageable surface and a secure and precisely positioned fit and positioning of the cutting guide to the patient's femur which thereby ensures proper positioning of the cutting guide surfaces of the cutting guide. The geometry of the patient-specific bone engaging surface is obtained from patient-specific images of the femur e.g., from X-rays, magnetic resonance imaging (MRI), CT scans, and the like. The patient-specific-images are used to create three dimensional (3D) images or models of the femur and then the patient-specific bone engaging surface is configured from the 3D models of the patient's femur.

Referring to FIGS. 2 and 3, the cutting guide 100 includes a first cutting guide surface 104, a second cutting guide surface 106, a third cutting guide surface 108, a fourth cutting guide surface 110, and a fifth cutting guide surface 112 formed on the non-metallic base. The first through fifth cutting guide surfaces are slotted formations configured to receive a suitable saw blade of a cutting tool for making bone cuts. While the present embodiment discloses slotted formations or slotted guides, the cutting guide can alternatively be formed with open faced cutting surfaces. The first, second, third, fourth, and fifth cutting guide surfaces are configured to make an anterior cut, an anterior chamfer cut, a distal cut, a posterior chamfer cut, and a posterior cut, respectively on the distal femur.

The positioning of the first, second, third, fourth, and fifth cutting guide surfaces relative to the overall geometry of the cutting guide, or the patient-specific bone engaging surface, are determined based on the patient-specific images of the patient's femur and corresponding 3D models of the patient's femur, and determined positions of the femur and tibia based on determined relative positions of 3D bone models for establishing joint balancing or a predetermined joint balancing of the patient's femur and tibia.

The cutting guide 100 further includes a medial opening or medial drill guide 114 and a lateral opening or lateral drill guide 116 about a distal end thereof. The medial opening 114 and the lateral opening 116 are distally facing openings or through opening that extend completely through the cutting guide. Each of the medial and lateral openings 114, 116 is sized sufficiently to receive a peg post of a femoral implant component such as the medial peg post 22 and the lateral peg post 24, respectively, of the femoral implant component 10, e.g., as shown in FIG. 1. The overall diameter of the medial and lateral openings 114, 116 can be e.g., 5 to 25 mm, including 6, 7, 8, 9, 10, 11, 12, 13, 14, 15, 16, 17, 18, 19, 20, 21, 22, 23, and 24 mm. Each of the medial and lateral openings 114, 116 is sized to be greater than an overall diameter of a drill pin used to pin the cutting guide to the patient's bone. Each of the medial and lateral openings 114, 116 is also positioned on the cutting guide to receive peg posts of a corresponding femoral implant or a planned femoral implant.

The cutting guide 100 further comprises medial and lateral gussets 118 and 120 which include cylindrical pin openings 122 and 124 for receiving retention pins 126. The retention pins 126 are inserted through the pin openings 122, 124 and drilled into the patient's bone to secure the cutting guide thereto. As shown in FIG. 3, the cutting guide may further include a proximally extending tongue or strap 128 having an opening 130 for receiving an unillustrated pin therein for further retaining the cutting guide to the patient's femur.

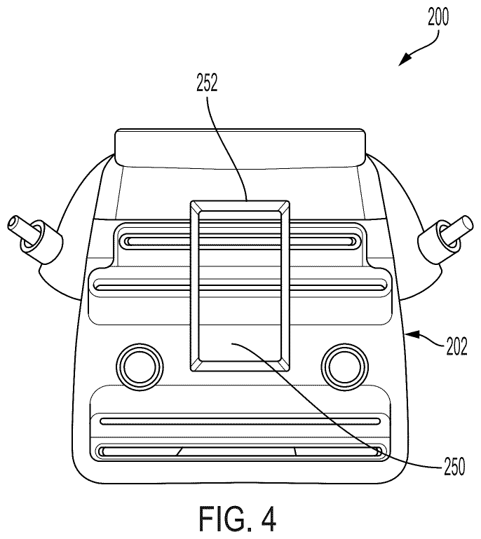

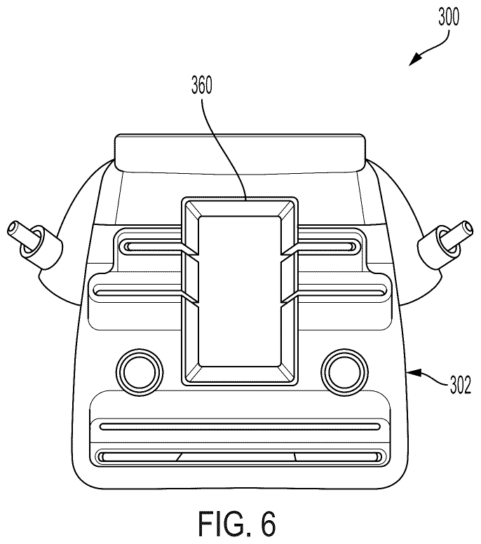

The patient-specific, non-metallic surgical cutting guide 100 or customized surgical cutting guide 100, as well as the patient-specific, non-metallic surgical cutting guides 200 and 300 of FIGS. 4 and 6, further discussed below, can be manufactured by non-metallic, e.g., plastic, material fabrication processes as described herein.

First, obtaining a patient-specific image or medical image of a patient's joint e.g., a knee joint, having a first bone e.g., a femur, and a second bone e.g., a tibia, and rendering 3D models or 3D bone models or computer models or computer aided design (CAD) models of the first and second bones of the joint. The rendering of 3D models of the joint can be accomplished through any number of computer aided orthopedic surgical (CAOS) systems implementing techniques well known in the art and for which a detailed description of such is not necessary for a full and complete understanding of the instant invention. Such techniques include e.g., obtaining medical images of a patient's bones e.g., computed tomography (CT) and using computer aided software to generate 3D models of the patient's bones based on the patient's medical images.

The CAOS then determines or determining relative positions of the 3D models of the first and second bones relative to each other for joint balancing. That is, the CAOS, which may receive additional inputs from a user, then determines a planned or optimal positioning of the first and second bones relative to each other to achieve a proper or planned joint balancing of the patient. Additional inputs from the user with respect to joint balancing can include information regarding e.g., the ACL (anterior cruciate ligament), the PCL (posterior cruciate ligament), the MCL (medial collateral ligament), and the LCL (lateral collateral ligament), and overall health and/or condition of such ligaments.

Once the optimal or planned position of the first and second bones are determined by e.g., the surgeon or user, the CAOS determines or determining the positions for a first bone cut, a second bone cut, a third bone cut, a fourth bone cut, and fifth bone cut to the first bone based on the determined relative positions of the 3D models of the first and second bones. The determined positions for the bone cuts are used to determine the positions on the various cutting guides surfaces on the cutting guide relative to a predetermined reference position e.g., the patient-specific bone engaging surface.

Based on the patient-specific information received from the patient-specific images and 3D models of the bone, the cutting guide or customized cutting guide can be manufactured. First, a CAD model of the customized cutting guide is created with the cutting guide model having a patient-specific bone engaging surface configured or modeled to correspondingly or matingly engage the patient's distal femur based on the 3D model of the first bone or femur. The cutting guide model is also configured to have the positions of the first cutting guide surface, the second cutting guide surface, the third cutting guide surface, the fourth cutting guide surface, and the fifth cutting guide surface each positioned on the non-metallic base based on the determined positions from patient-specific 3D bone models of a patient for the first bone cut, the second bone cut, the third bone cut, the fourth bone cut, and the fifth bone cut, and/or the bone engaging surface of the cutting guide.

The method of manufacturing the patient-specific cutting guide further comprises manufacturing a non-metallic surgical guide having a non-metallic base including a first cutting guide surface, a second cutting guide surface, a third cutting guide surface, a fourth cutting guide surface and a fifth cutting guide surface each positioned on the non-metallic base based on the determined positions from patient-specific 3D bone models of a patient for the first bone cut, the second bone cut, the third bone cut, the fourth bone cut, and the fifth bone cut, a medial opening about its distal end sized sufficiently to receive a peg post of a femoral implant component, and a lateral opening about its distal end sized sufficiently to receive a peg post of a femoral implant component.

The customized cutting guide can then be manufactured using any non-metallic fabrication process e.g., via a 3D printing process, or any other process that allows for the cutting guide to be manufactured based on the cutting guide models. Suitable plastics usable in the 3D printing process can include, without limitation, polyethylene terephthalate (PET), polyethylene terephthalate glycol (PETG), polycarbonate (PC), polypropylene (PP), nylon, polyetheretherketone (PEEK), polyetherketoneketone (PEKK), polyetherimide (PEI), acrylonitrile styrene acrylate (ASA), acrylonitrile butadiene styrene (ABS) and polylactic acid (PLA).

In accordance with further exemplary embodiments of the subject disclosure, there are shown in FIGS. 4 and 6 embodiments of non-metallic surgical cutting guides 200 and 300. The non-metallic surgical cutting guides 200 and 300 correspond in many ways to the structure and function of the non-metallic surgical cutting guide 100 of FIGS. 2 and 3. Accordingly, only those aspects of the non-metallic surgical cutting guides 200 and 300 which materially depart in structure and/or function from their counterparts in FIGS. 2 and 3, or are otherwise necessary for a proper understanding of the subject disclosure, will be described in detail.

FIG. 4 illustrates a second exemplary embodiment of a non-metallic surgical cutting guide 200 comprising a detachable center portion 250 which is detachable or removable from the cutting guide base e.g., after making the anterior cut, the anterior chamber cut, the distal cut, the posterior chamber cut and the posterior cut on the patient's femur. The detachable center portion 250 can be connected to the cutting guide base e.g., via fasteners, or brackets, or magnetically, or via any other suitable connection as known in the art.

The detachable center portion 250 is sized sufficiently such that when removed from the cutting guide base a through hole 252 remains sized sufficiently to receive a femoral preparation instrument such as a femoral punch (not shown) therethrough. Such femoral punches are well known in the art along with their structure and operation in connection with total knee arthroplasty surgeries.

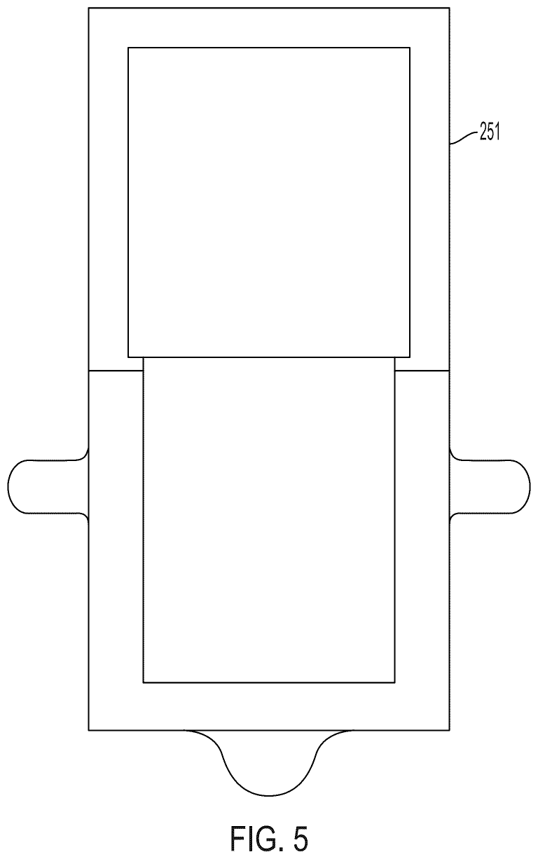

Alternatively, the cutting guide 200 can include a femoral punch guide 251 (FIG. 5). The femoral punch guide 251 is sized and configured to seat with the through hole of the cutting guide after the detachable center portion is removed. The femoral punch guide 251 then serves as a guide for receiving the femoral punch instrument therethrough for preparing or forming a distal femoral notch.

Referring to FIG. 6, the non-metallic surgical cutting guide 300 comprises a central opening 360 sized sufficiently to receive a femoral preparation instrument such as a femoral punch (not shown) therethrough, but not so large as to compromise the ability to accurately make bone cuts through the first through fifth cutting guide surfaces.

Each of the non-metallic surgical cutting guides 100, 200 and 300 disclosed herein provides, in a single device, a guide that enables the anterior cut, the anterior oblique cut, the distal cut, the posterior oblique cut and the posterior cut to be made at the patient's distal femur, as well as distal medial and lateral bores to be drilled in the distal face of the cut femur without need for an additional drill guide. Further, the non-metallic surgical cutting guides of the subject disclosure can be used in connection with cruciate retaining or posterior stabilized knee replacement implants.

It will be appreciated by those skilled in the art that changes could be made to the various aspects described above without departing from the broad inventive concept thereof. It is to be understood, therefore, that the subject application is not limited to the particular aspects disclosed, but it is intended to cover modifications within the spirit and scope of the subject application as defined by the appended claims.

I claim:

1. A customized surgical cutting guide comprising:
a non-metallic base having a patient-specific bone engaging surface;
a first cutting guide surface, a second cutting guide surface, a third cutting guide surface, a fourth cutting guide surface and a fifth cutting guide surface formed on the non-metallic base;
a medial opening about a distal end of the non-metallic base sized sufficiently to receive a peg post of a femoral implant component; and
a lateral opening about the distal end of the non-metallic base sized sufficiently to receive a peg post of a femoral implant component.

2. The customized surgical cutting guide of claim 1, wherein the first cutting guide surface, the second cutting guide surface, the third cutting guide surface, the fourth cutting guide surface, and the fifth cutting guide surface are positioned on the non-metallic base based on patient-specific 3D bone models of a patient.

3. The customized surgical cutting guide of claim 1, wherein the non-metallic base includes a detachable center portion.

4. The customized surgical cutting guide of claim 3, wherein the detachable center portion is sized sufficiently to receive a femoral preparation instrument therethrough.

5. The customized surgical cutting guide of claim 3, further comprising a femoral punch guide for replacing the detachable center portion.

6. The customized surgical cutting guide of claim 1, wherein the non-metallic base is a unitarily formed base.

7. The customized surgical cutting guide of claim 1, wherein the medial and lateral openings are distally facing openings.

8. The customized surgical cutting guide of claim 1, wherein the non-metallic base comprises a polymer.

9. The customized surgical cutting guide of claim 1, wherein the non-metallic base comprises polyethylene terephthalate (PET), polyethylene terephthalate glycol (PETG), polycarbonate (PC), polypropylene (PP), nylon, polyetheretherketone (PEEK), polyetherketoneketone (PEKK), polyetherimide (PEI), acrylonitrile styrene acrylate (ASA), and/or acrylonitrile butadiene styrene (ABS) and polylactic acid (PLA).

10. A method of manufacturing a patient-specific, non-metallic surgical cutting guide comprising:
obtaining a medical image of a first bone and a second bone of a joint of a patient;
rendering 3D models of the first and second bones of the joint based on the medical image;
determining relative positions of the 3D models of the first and second bones relative to each other for establishing joint balancing;
determining positions for a first bone cut, a second bone cut, a third bone cut, a fourth bone cut, and a fifth bone cut to the first bone based on the determined relative positions of the 3D models of the first and second bones; and
manufacturing a non-metallic surgical cutting guide having:
a bone engaging surface based on the rendered 3D model of the first bone,
a first cutting guide surface, a second cutting guide surface, a third cutting guide surface, a fourth cutting guide surface and a fifth cutting guide surface each positioned based on the determined positions for the first bone cut, the second bone cut, the third bone cut, the fourth bone cut, and the fifth bone cut,
a medial opening about its distal end sized sufficiently to receive a peg post of a femoral implant component, and
a lateral opening about its distal end sized sufficiently to receive a peg post of a femoral implant component.

11. The method of claim 10, wherein the non-metallic surgical cutting guide further includes a detachable center portion.

12. The method of claim 10, wherein manufacturing the non-metallic surgical cutting guide includes 3D printing the surgical cutting guide.

13. A surgical cutting guide comprising:
a base having a bone engaging surface;
a first cutting guide surface, a second cutting guide surface, a third cutting guide surface, a fourth cutting guide surface and a fifth cutting guide surface formed on the base;
a medial opening about a distal end of the non-metallic base sized sufficiently to receive a peg post of a femoral implant component; and
a lateral opening about the distal end of the non-metallic base sized sufficiently to receive a peg post of a femoral implant component.

14. The surgical cutting guide of claim 13, wherein the base includes a detachable center portion.

15. The surgical cutting guide of claim 14, wherein the detachable center portion is sized sufficiently to receive a femoral preparation instrument therethrough.

16. The surgical cutting guide of claim 14, further comprising a femoral punch guide for replacing the detachable center portion.

17. The surgical cutting guide of claim 13, wherein the medial and lateral openings are distally facing openings.
